SEQUENCE LISTING (36 sequences)

SEQ ID NO: 1 - protein, 5 aa, Artificial Sequence
  The sequence is produced recombinantly, e.g. in cell lines, and is thus man-made
NYGVH

SEQ ID NO: 2 - protein, 16 aa, Artificial Sequence
  The sequence is produced recombinantly, e.g. in cell lines, and is thus man-made
VIWSGGNTDYNTPFTS

SEQ ID NO: 3 - protein, 11 aa, Artificial Sequence
  The sequence is produced recombinantly, e.g. in cell lines, and is thus man-made
ALTYYDYEFAY

SEQ ID NO: 4 - protein, 11 aa, Artificial Sequence
  The sequence is produced recombinantly, e.g. in cell lines, and is thus man-made
RASQSIGTNIH

SEQ ID NO: 5 - protein, 6 aa, Artificial Sequence
  The sequence is produced recombinantly, e.g. in cell lines, and is thus man-made
ASESIS

SEQ ID NO: 6 - protein, 9 aa, Artificial Sequence
  The sequence is produced recombinantly, e.g. in cell lines, and is thus man-made
QQNNNWPTT

SEQ ID NO: 7 - protein, 119 aa, Artificial Sequence
  The sequence is produced recombinantly, e.g. in cell lines, and is thus man-made
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 107 aa, Artificial Sequence
  The sequence is produced recombinantly, e.g. in cell lines, and is thus man-made
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 9 - protein, 449 aa, Artificial Sequence
  The sequence is produced recombinantly, e.g. in cell lines, and is thus man-made
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  The sequence is produced recombinantly, e.g. in cell lines, and is thus man-made
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 11 - protein, 107 aa, Artificial Sequence; identical to SEQ ID NO: 8
  The sequence is produced recombinantly, e.g. in cell lines, and is thus man-made

SEQ ID NO: 12 - protein, 107 aa, Artificial Sequence
  The sequence is produced recombinantly, e.g. in cell lines, and is thus man-made
DILLTQSPVILSVSPGERVSFSCRASQSIGTNIHWYQQRTNGSPRLLIKYASKSISGIPSRFSGSGSGTDFTLSINSVESEDIADYYCQQNNNWPTTFGAGTKLELK

SEQ ID NO: 13 - protein, 119 aa, Artificial Sequence
  The sequence is produced recombinantly, e.g. in cell lines, and is thus man-made
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYADYEFAYWGQGTLVTVSA

SEQ ID NO: 14 - protein, 119 aa, Artificial Sequence
  The sequence is produced recombinantly, e.g. in cell lines, and is thus man-made
QVQLKQSGPGLVQPSQSLSITCTVSGFSLTNYGVHWVRQSPGKGLEWLGVIWSGGNTNYNTPFTSRLSINKDNSKSQVFFKMNSLQSNDTAIYYCARALTYYNYEFAYWGQGTLVTVSA

SEQ ID NO: 15 - protein, 119 aa, Artificial Sequence
  The sequence is produced recombinantly, e.g. in cell lines, and is thus man-made
QVQLKQSGPGLVQPSQSLSITCTVSGFSLTNYGVHWVRQSPGKGLEWLGVIWSGGNTNYNTPFTSRLSINKDNSKSQVFFKMNSLQSNDTAIYYCARALTYYNYQFAYWGQGTLVTVSA

SEQ ID NO: 16 - protein, 16 aa, Artificial Sequence
  The sequence is produced recombinantly, e.g. in cell lines, and is thus man-made
RSSQNIVHSNGNTYLD

SEQ ID NO: 17 - protein, 7 aa, Artificial Sequence
  The sequence is produced recombinantly, e.g. in cell lines, and is thus man-made
KVSNRFS

SEQ ID NO: 18 - protein, 9 aa, Artificial Sequence
  The sequence is produced recombinantly, e.g. in cell lines, and is thus man-made
FQYSHVPWT

SEQ ID NO: 19 - protein, 5 aa, Artificial Sequence
  The sequence is produced recombinantly, e.g. in cell lines, and is thus man-made
NYYIY

SEQ ID NO: 20 - protein, 17 aa, Artificial Sequence
  The sequence is produced recombinantly, e.g. in cell lines, and is thus man-made
GINPTSGGSNFNQKFKT

SEQ ID NO: 21 - protein, 14 aa, Artificial Sequence
  The sequence is produced recombinantly, e.g. in cell lines, and is thus man-made
QGLWFDSDGRGFDF

SEQ ID NO: 22 - protein, 455 aa, Artificial Sequence
  The sequence is produced recombinantly, e.g. in cell lines, and is thus man-made
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, Artificial Sequence
  The sequence is produced recombinantly, e.g. in cell lines, and is thus man-made
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 122 aa, Artificial Sequence
  The sequence is produced recombinantly, e.g. in cell lines, and is thus man-made
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVS

SEQ ID NO: 25 - protein, 111 aa, Artificial Sequence
  The sequence is produced recombinantly, e.g. in cell lines, and is thus man-made
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIKRTVA

SEQ ID NO: 26 - protein, 118 aa, Artificial Sequence
  The sequence is produced recombinantly, e.g. in cell lines, and is thus man-made
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVS

SEQ ID NO: 27 - protein, 114 aa, Artificial Sequence
  The sequence is produced recombinantly, e.g. in cell lines, and is thus man-made
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIKRT

SEQ ID NO: 28 - protein, 113 aa, Artificial Sequence
  The sequence is produced recombinantly, e.g. in cell lines, and is thus man-made
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKR

SEQ ID NO: 29 - protein, 121 aa, Artificial Sequence
  The sequence is produced recombinantly, e.g. in cell lines, and is thus man-made
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSA

SEQ ID NO: 30 - protein, 170 aa, Artificial Sequence
  The sequence is produced recombinantly, e.g. in cell lines, and is thus man-made
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSASTKGPSVFPLAPSSKSTSGGTAALGCLVKDYFPEPVTVSWNSGALTSGV

SEQ ID NO: 31 - protein, 110 aa, Artificial Sequence
  The sequence is produced recombinantly, e.g. in cell lines, and is thus man-made
DTVMTQTPLSSHVTLGQPASISCRSSQSLVHSDGNTYLSWLQQRPGPPRLLIYRISRRFSGVPDRFSGSGAGTDFTLEISRVEAEDVGVYYCMQSTHVPRTFGQTKVEIK

SEQ ID NO: 32 - protein, 124 aa, Artificial Sequence
  The sequence is produced recombinantly, e.g. in cell lines, and is thus man-made
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSS

SEQ ID NO: 33 - protein, 124 aa, Artificial Sequence; identical to SEQ ID NO: 32
  The sequence is produced recombinantly, e.g. in cell lines, and is thus man-made

SEQ ID NO: 34 - protein, 134 aa, Artificial Sequence
  The sequence is produced recombinantly, e.g. in cell lines, and is thus man-made
MRVLILLWLFTAFPGVLSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 35 - protein, 128 aa, Artificial Sequence
  The sequence is produced recombinantly, e.g. in cell lines, and is thus man-made
MVSTAQFLAFLLLWFPGARCDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 36 - protein, 14 aa, Artificial Sequence
  The sequence is produced recombinantly, e.g. in cell lines, and is thus man-made
LEEKKGNYVVTDHC

(19) United States
(12) Patent Application Publication
(10) Pub. No.: US 2017/0333570 A1
(43) Pub. Date: Nov. 23, 2017

(54) EGFR ANTIBODY-BASED COMBINATION THERAPY

(71) Applicant: Formation Biologics Inc., Toronto (CA)

(72) Inventor: Ilia Alexandre Tikhomirov, Houston, TX (US)

(21) Appl. No.: 15/522,975

(22) PCT Filed: Oct. 30, 2015
(86) PCT No.: PCT/CA2015/000557

§ 371 Date: Apr. 28, 2017

Related U.S. Application Data
(60) Provisional application No. 62/073,380, filed on Oct. 31, 2014.

(51) Int. Cl. A61K 47/68; A61K 39/395; C07K 16/28; A61K 47/00

(52) U.S. Cl. CPC A61K 47/6817; C07K 16/2863; A61K 47/6849; A61K 39/395; A61K 47/00

(57) ABSTRACT

EGFR antibodies can be combined to enhance their anti-cancer effect. In the present invention, at least one of two naked antibodies in a combination is provided as an antibody drug conjugate (ADC) in which the antibody is coupled with a cytotoxin. Both antibodies also can be provided in ADC form. The result is improved activity, with the improvement being synergistic in many such antibody pairs.

### FIELD OF THE INVENTION

The present invention relates to medical treatments and antibody-based treatment combinations that affect EGFR+ cancer cells and tumours comprising them.

### BACKGROUND TO THE INVENTION

Epidermal growth factor receptor, or EGFR, is an attractive target for the development of anti-cancer antibodies and immunoconjugates (antibody drug conjugates, or ADCs) because of the antigen's expression by many tumors and its rapid internalization. However, because EGFR is also expressed by skin tissues, EGFR-targeting agents, such as the antibodies cetuximab and panitumumab, also show levels of skin toxicities that either demand dose reduction or in some cases are so severe as to warrant discontinuation of treatment.

Anti-EGFR immunoconjugates are now being designed specifically to address these safety concerns. These conjugates are based on antibodies that target a mutated but naturally occurring version of EGFR, known as EGFRvIII, or on conformational forms of the EGFR, both of which predominate on tumour cells and not on skin cells

(U.S. Pat. No. 7,628,986 and U.S. Pat. No. 7,589,180, respectively). For example, anti-EGFR antibody MAb806 is an antibody that targets an EGFR epitope found only on cancer cells, and potentially offers an advantage over the current EGFR antibodies, which all display significant binding to normal organs such as skin in humans. An immunoconjugate comprising EGFR MAb 806 linked to an anti-microtubule payload is currently in phase I clinical testing in patients with advanced solid tumours.

Efforts continue through screening for naked anti-EGFR antibodies, and immunoconjugates thereof, to identify those with partial antagonistic activity against EGFR and reduced activity against keratinocytes (see US 2012/0156217), for immunoconjugates based on “masked” anti-EGFR antibodies that are preferentially activated in the tumour microenvironment (WO 2009/025846), and for immunoconjugates based on antibodies with medium affinity that preferentially accumulate in the tumor and not normal tissues (WO 2012/100346).

The therapeutic effects of antibody combinations have also been studied, using compositions comprising at least two different anti-EGFR antibody species, where each species binds simultaneously to EGFR at distinct epitopes. For example, Friedmann et al (Proc. Natl. Acad. Sci., 2005, 102:1915-20) show that two murine monoclonal antibodies selected for their ability to inhibit EGF binding to EGFR by binding distinct EGFR epitopes are able to synergistically down-regulate receptor expression in KB cells and CHO cells transiently expressing EGFR. Cross competitive EGF inhibiting antibodies did not exhibit any synergy.

Modjtahedi et al (Cell Biophysics vol 22, 1993, 129-146) has tested combinations of several rat anti-EGFR antibodies that bind at non-overlapping epitopes. The antibodies were of different isotypes as well. In all cases, the effect of using two antibodies was intermediate between the effects of using similar amounts of the two monoclonal antibodies alone. This was confirmed both in vivo and in vitro.

Merck's WO 2004/032960 discloses that the combined use of two monoclonal antibodies, Mab425 and Mab225 (Cetuximab), results in an increased amount of antibodies bound to the surface of EGFR expressing cancer cells compared to a similar amount of each of the monoclonal antibodies alone. The publication also discloses increased down-regulation of EGFR when using the combination of antibodies compared to the two monoclonal antibodies separately.

Perera et al (Clin Cancer Res 2005; 11 (17):6390-99) disclosed a synergistic effect of treating mice bearing U87MG.de2-7 xenografts with a combination of two murine monoclonal antibodies. One of the antibodies (mAb 528) binds all of the EGFR subtypes with similar specificity to cetuximab. The other antibody (mAB 806) only binds the de2-7 EGFR. The U87MG.de2-7 cell line is a de2-7EGFR transfected cell line. The U87MG.DK cell line expresses a kinase inactive variant of the de2-7 EGFR. No synergy was observed when the two antibodies were used against mice bearing U87MG.DK xenografts. In a xenograft model with the A431 cell line expressing wildtype EGFR, the authors provided no evidence of synergy. The de2-7 EGFR is only present in a limited number of cancer types, such as glioma, to some extent breast cancer and lung cancers.

In U.S. Pat. No. 7,887,805, Symphogen describes naked EGFR antibody mixtures that are more potent against cancer cell lines than individual antibodies. The Symphogen mixtures comprise at least 2, and up to 3, different EGFR antibody species.

The above publications and patents describe mixtures of naked anti-EGFR antibodies. Though these mixtures are frequently more potent than the individual anti-EGFR antibodies comprising the mixtures, the mixtures still exhibit variability in their effects on different cancer cell lines, and resistance is common.

Accordingly, the need exists for improved therapeutic anti-EGFR targeting approaches which are effective at treating and/or preventing diseases related to overexpression of EGFR.

An object of the present invention is to provide a drug combination that is useful to treat EGFR+ disease cells including EGFR+ cancer cells and tumours comprising them. Another object of the present invention is to provide a method for enhancing the cytotoxicity of a given EGFR antibody or a given EGFR antibody combination toward disease cells.

### SUMMARY OF THE INVENTION

EGFR antibodies that cooperate to produce an anti-cancer effect on EGFR+ disease cells are used in an altered combination wherein at least one of the EGFR antibody species is provided as an EGFR antibody drug conjugate. The result is an EGFR antibody combination having a superior and even synergistic effect on the killing of targeted EGFR+ disease cells.

Thus, in one of its aspects, the present invention provides a pharmaceutical combination, the combination comprising at least two different species of human EGFR antibody, wherein all such antibody species will bind simultaneously to human EGFR and further wherein at least one of such antibodies is provided as an antibody drug conjugate. In preferred embodiments, the combination provides an effect on killing of EGFR+ disease cells that is synergistic, relative to a combination of naked EGFR antibodies that lacks any species of antibody drug conjugate.

In another of its aspects, there is provided a process for preparing an antibody composition useful to treat EGFR+ disease cells, comprising mixing first and second EGFR antibodies, wherein at least one of said antibodies is an EGFR antibody drug conjugate.

In another aspect, there is provided a method for treating a subject presenting with EGFR+ disease cells, the method comprising administering to the subject a pharmaceutical combination comprising at least two different species of human EGFR antibody, wherein all such antibodies will bind simultaneously to human EGFR and further wherein at least one of such antibodies is provided as an antibody drug conjugate, the combination providing an effect on killing of EGFR+ disease cells that preferably is synergistic, relative to a combination lacking any species of antibody drug conjugate. In a related aspect, there is provided the use, for controlling the growth of EGFR+ disease cells, of a pharmaceutical combination comprising at least two EGFR antibody species that will bind EGFR simultaneously, wherein at least one of said species is provided as an EGFR antibody-drug conjugate, and further wherein the activity of the combination is synergistic with respect to the killing of said disease cells, relative to the activity of a combination comprising the same two antibodies as naked EGFR antibodies.

These and other aspects of the present invention are now described in greater detail with reference to the accompanying drawings in which:

### DESCRIPTION OF THE DRAWINGS

FIG. 1 is a line graph depicting the cytotoxic activity of the indicated antibody-drug conjugates and 1:1 mixture of antibody-drug conjugates against A549 cancer cell line. Synergistic activity of the antibody-drug conjugates mixture is observed.

FIG. 2 is a line graph depicting the cytotoxic activity of the indicated antibody-drug conjugates and 1:1 mixture of antibody-drug conjugates against NCI-H292 cancer cell line. Synergistic activity of the antibody-drug conjugates mixture is observed.

FIG. 3 is a line graph depicting the cytotoxic activity of the indicated antibody-drug conjugates and 1:1 mixture of antibody-drug conjugates against NCI-H226 cancer cell line. Synergistic activity of the antibody-drug conjugates mixture is observed.

FIG. 4 is a line graph depicting the cytotoxic activity of indicated antibodies, antibody-drug conjugates and mixture of indicated antibody and antibody-drug conjugates against NCI-H292 cancer cell line. Synergistic activity of the antibody-drug conjugate plus antibody mixture is observed.

### DETAILED DESCRIPTION AND PREFERRED EMBODIMENTS

The present invention relates to antibodies and immunoconjugates that bind to the human epidermal growth factor receptor (hEGFR), a protein that is presented on the surface of many different cell types including pancreas, lung, ovaries, kidney, GI tract, and brain, among others. As used herein, the term “hEGFR” refers to any protein that comprises the expressed and processed product of the human her-1 gene, wherein the protein is designated as UniProtKB/Swiss-Prot P00533. The term EGFR is used generically herein, and refers to the wild type protein and all naturally occurring variants thereof. The term “wtEGFR” is used more specifically with reference only to the wild type form of human EGFR. The term “EGFRvIII” refers to the EGFR variant protein that comprises the expressed and processed product of a variant of the her-1 gene lacking exons 2-7, and thus includes only the polypeptide sequence encoded by exons 1 and 8 of her-1. The term “domain III” is not related to EGFRvIII, and instead refers to a location within EGFR, and represents an extracellular site that is key for EGF ligand binding, and binding of highly antagonistic antibodies cetuximab and panitumumab (Voigt et al, 2012 November; 14(11): 1023-1031).

The EGFR antibodies useful in the present combination are those that bind to non-overlapping epitopes of EGFR and can promote internalization of the receptors as a mixture. The antibodies thus incorporate binding sites for any unique epitope of human EGFR. The “binding sites” or antigen binding “domains” exploited by the present invention are the sites within EGFR antibodies that bind to EGFR. An “antigen binding site” of these antibodies is defined as the antibody domain that specifically binds to and is complementary to part of or the entire target antigen. An antigen binding site may be provided by one or more antibody variable domains. Preferably, an antigen binding site comprises the antigen-binding combination of an antibody light chain variable region (VL) and an antibody heavy chain variable region (VH). In the antibodies used in the present combination, each such antibody and any target binding fragment thereof comprises at least one EGFR binding site or binding domain, and will usually comprise two such sites or domains. In embodiments, the two such sites or domains bind the same type of antigen binding site, and more usually they have the same amino acid sequence in their complementarity determining regions (CDRs). In the alternative, the antibodies and their binding fragments can be bispecific antibodies, and will therefore present two different EGFR binding sites. They are of any isotype that is suitable for human use without eliciting an adverse immune response, including IgG1, IgG2 and IgG4, and typically any of IgA, IgE, and more likely IgM or IgG.

The present antibody combinations are those in which all of the antibodies in the combination will bind simultaneously to the EGFR target so that the effect of each antibody is exerted and not blocked. This can be tested using a variety of assays. For instance, using BIA-CORE analysis, one of the EGFR antibodies is immobilized on the surface of the chip. As EGFR is injected into the system, the resonance signal increases as the EGFR and immobilized antibody associate. Another antibody (selected purposively or randomly) is then injected. If the injected antibody and the immobilized antibody compete for binding, then there will be no signal coming from the binding of the second. If the injected antibody and the immobilized antibody can bind simultaneously, then there will be a signal coming from the binding of the second antibody.

In some embodiments of the present invention, the EGFR binding sites are found in, e.g., “derived from”, existing “parental” EGFR antibodies especially those EGFR antibodies that are either approved for marketing or are in clinical development. Thus, in embodiments and for convenience, the antibodies used in combination either are, or comprise, EGFR binding sites that are present in parental EGFR antibodies that include cetuximab, cetuximab variants with comparable or reduced EGFR affinity, panitumumab, nimotuzumab, zalutumumab, matuzumab, and the like. The actual sequences of the EGFR binding sites for some of these antibodies are provided in the listed sequences that appear at the end of this disclosure, or have sequences reported in the literature cited here, and are summarized below:
Cetuximab (Erbitux®)
Cetuximab variants described in WO 2012/100346
Panitumumab (Vectibix®)
Necitumumab
Zalutumumab
Matuzumab (see Kelton et al, Arch. Biochem. Biophys., 2012, 526:219-225)
Nimotuzumab
ch806
13.1
13.1.2
1024 (see U.S. Pat. No. 7,887,805)
992 (see U.S. Pat. No. 7,887,805)
1030 (see U.S. Pat. No. 7,887,805)
111 (CNCM deposit number 1-4261) see Spangler et al, PNAS, 2010, 107(30):13253), and Yeda U.S. Pat. No. 7,939,072
565 (CNCM deposit number 1-4262) see Spangler et al, above), and see Yeda's US′072
Antibody species within so-called MM-151 (see Tan et al, AACR/EORTC 2011 poster A210 for mechanism); and
species J2898A.

The antibody species within the SYM004 mixture comprise at least two of the species 992, 1024 and 1030 as noted above and described in U.S. Pat. No. 7,887,805 and by Pedersen et al in Cancer Res. Jan. 15 2010, 70(2), both incorporated herein by reference.

The antibody mixture known as MM-151 comprises 3 naked EGFR antibodies that include those antibodies described in Merrimack's U.S. Pat. No. 9,044,460, i.e., EGFR antibodies designated ca, cd and ch. The sequences of the CDRs for each antibody are provided in the US'460 patent, and are incorporated herein by reference.

Cetuximab is a recombinant, human/mouse chimeric IgG1 antibody that binds specifically to the extracellular domain of wtEGFR. The amino acid sequences of the CDRs for both the heavy chain of cetuximab (SEQ ID Nos.1-3) and the light chain of cetuximab (SEQ ID Nos. 4-6) are listed herein. Also listed are the amino acid sequences of the heavy chain variable region (SEQ ID No.7) and of the light chain variable region (SEQ ID No.8) of cetuximab, together with the amino acid sequences of the complete heavy chain (SEQ ID No. 9) and complete light chain (SEQ ID No.10) of cetuximab.

The useful EGFR antibody can be a full antagonist at EGFR or a partial antagonist thereat. An EGFR antibody that is a “full antagonist” is an antibody that blocks completely or nearly so the transmission of a signal that is stimulated, in the normal course, by the EGF ligand through EGFR to the EGFR-coupled tyrosine kinase. EGFR antibodies that are full antagonists are particularly EGFR antibodies that bind directly to EGFR domain III. EGFR antibodies having these properties and an EGFR binding affinity of 5 nanomolar (nM) or less are particularly preferred for inclusion in the present combination, either as naked antibodies or as antibody drug conjugates.

It should be understood that the antibodies useful in the present invention include antibodies that bind to the same epitope as those antibodies identified specifically herein. Those antibodies will have a different sequence in their antigen binding sites yet will compete for EGFR binding with the corresponding known antibody.

The present immunoconjugates (or antibody drug conjugates, ADCs) can be based more particularly, and in one embodiment, on the hEGFR antibody species known as cetuximab, now commercially available under the trade name Erbitux®.

Cetuximab variants are also useful herein for binding to human EGFR. Useful cetuximab variants include fragments of cetuximab comprising the EGFR binding sites of cetuximab, such as all of the light chain and heavy chain CDRs herein recited. Other cetuximab variants useful herein are cetuximab variants that incorporate one, two or more substitutions outside the antigen binding domains, such as in the framework region or in the constant region (Fc). Such substitutions are benign in the sense that they do not reduce cytotoxicity relative to cetuximab per se. Cetuximab variants include those in which the light chain variable region has SEQ ID No. 10 or 11 and the heavy chain variable region is SEQ ID No.12, 13 or 14.

In another embodiment, the EGFR antibody species can be panitumumab, now commercially available and sold under the trade name Vectibix®, or an ADC thereof. Panitumumab is a recombinant, fully human IgG2 antibody that binds specifically to the extracellular domain of wtEGFR. The amino acid sequences of the heavy and light chains of panitumumab are listed in U.S. Pat. No. 6,235,883 and U.S. Pat. No. 7,807,798, incorporated herein by reference. A useful alternative to panitumumab is an EGFR binding variant thereof that competes with panitumumab for EGFR binding, such as a fragment of panitumumab that incorporates its antigen binding sites but has an otherwise lost or altered constant region.

The present ADCs can also be based on still other EGFR antibody species provided they show EGFR antagonist activity, such as EGFR antibody species that bind selectively to domain III of EGFR, and any other EGFR antibodies that compete with EGF and block fully or nearly so the transmission of EGF-stimulated downstream signalling.

In the present antibody combination, at least one of the EGFR antibody species in the pair, such as a pair including cetuximab or panitumumab, is conjugated to any desired cytotoxin. This includes anti-microtubule toxins such as maytansinoid toxin. By “anti-microtubule toxin” is meant an agent having cell toxicity mediated by interference with the microtubule structures important for cell mitosis, such as by inhibiting the formation of tubulin or by inhibiting the organization thereof.

Included within this toxin family are the maytansinoids and auristatins, and many other agents developed more recently and having the same mechanism of action. The auristatins in particular block cell replication by inhibiting polymerization of tubulin and are thus anti-mitotic. The structure of an auristatin useful herein and known as MMAE, or vedotin, is shown below:

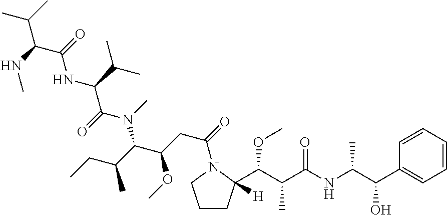 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

There are various forms of maytansinoids that are useful. These are all based on the complex structure of a natural molecule, maytansine.

Particularly useful are the maytansinoids including DM-1 and DM-4. In a specific embodiment, the toxin coupled to the EGFR MAb is DM-1 having the structure shown infra.

Also useful as anti-microtubule toxins are dolostatins, auristatins, tubulysins and cryptophycins. Specific examples of useful species within each genus include dolostatin 10, monomethyl dolostatin 10, auristatin E, monomethyl auristatin E (MMAE), auristatin F, monomethyl auristatin F, HTI-286, tubulysin M, as well as the tubulin binders such as tubulysin IM-1, tubulysin IM-2, tubulysin IM-3, colchicine DA, and maytansinoids AP-3, DM-1 and DM-4.

Conjugates of an EGFR antibody such as cetuximab or panitumumab, and an anti-microtubule toxin such as a maytansinoid or auristatin can be formed using any technique presently known or later developed that couples a linker, such as a linker that is “non-cleavable”. These linkers remain intact, and retain the antibody and toxin in covalent association, throughout conditions normally encountered following administration to a subject, including extracellular environments. More specifically, non-cleavable linkers result in ADC constructs for which release of the cytotoxic payload is achieved by destruction of the antibody by intracellular lysosomes.

Methods of linker integration are described for instance in U.S. Pat. No. 5,208,020; U.S. Pat. No. 8,088,387; and U.S. Pat. No. 6,441,163. A preferred method is to modify the EGFR antibody, e.g., cetuximab, with succinimidyl 4-(N-maleimidomethyl)-cyclohexane-1-carboxylate (SMCC) to introduce maleimido groups followed by reaction of the modified antibody with a thiol-containing maytansinoid to give a thioether-linked conjugate. Conjugates with 1 to 10 drug molecules per antibody molecule will result.

Other useful forms of non-cleavable linkers include N-Succinimidyl iodoacetate (SIA), sulfo-SMCC, m-maleimidobenzoyl-N-hydroxysuccinimide ester (MBS), sulfo-MBS and succinimidyl-iodoacetate, as described in the literature, to introduce 1-10 reactive groups. (see, Yoshitake et al, 101 Eur. J. Biochem. 395-399 (1979); Hashida et al, J. Applied Biochem. 56-63 (1984); and Liu et al, 18 690-697 (1979)). Particularly useful for linking auristatins as anti-microtubule toxin are the non-cleavable maleimidocaproyl linkers described by Doronina et al, in Bioconjugate Chem., 2006 January-February; 17(1):114-24).

In a specific embodiment, the drug combination comprises at least the immunoconjugate that is cetuximab linked to DM-1 by an SMCC linker.

In another specific embodiment, the drug combination comprises at least the immunoconjugate that is panitumumab linked to DM-1 by an SMCC linker.

In another specific embodiment, the drug combination comprises a naked anti-EGFR antibody that is cetuximab.

In another specific embodiment, the drug combination comprises a naked anti-EGFR antibody that is panitumumab.

In a specific embodiment, the drug combination comprises at least the immunoconjugate that is 992 linked to DM-1 by an SMCC linker.

In another specific embodiment, the drug combination comprises at least the immunoconjugate that is 1024 linked to DM-1 by an SMCC linker.

In another specific embodiment, the drug combination comprises a naked anti-EGFR antibody that is 992.

In another specific embodiment, the drug combination comprises a naked anti-EGFR antibody that is 1024.

In more specific embodiments the drug combination comprises naked 992 and conjugated 1024. Alternatively the drug combination comprises naked 1024 and conjugated 992. In a further specific embodiment, the drug combination comprises conjugated 992 and conjugated 1024.

In another specific embodiment, the drug combination is related with the MM-151 mixture, where at least one of EGFR antibodies ca, cd and ch is conjugated with a cytotoxin that is desirably DM-1 through a linker that is desirably SMCC.

In other specific embodiments, the antibody composition comprises at least two of the 4 antibody types just recited, wherein at least one of them is an ADC.

The antibody combinations described herein are characterized as being synergistic or as displaying synergism. This means that the two or more antibodies have an activity that is greater when they are combined than the expected additive effect of the individual naked antibody activities or the combined naked antibody activities. The activity used for this comparison can be any of the EGF activities that are expected to be altered by the EGFR antibodies. For instance and as used herein, the activity tested can be the antibody effect on the growth rate of EGFR+ cancer cells, as discussed infra.

Thus, in a general aspect, the invention provides a method that potentiates the anti-cancer activity of a naked EGFR antibody combination, by replacing at least one of the naked antibodies with a counterpart to which is conjugated a cytotoxin effective against EGFR+ disease cells. When combined, the EGFR antibody drugs have a synergistic anti-cancer activity with respect to survival of EGFR+ disease cells, relative to combinations that contain only naked EGFR antibodies.

In embodiments of the present invention, the antibody combination is selected from an EGFR antibody pair in in which at least one or both of the antibodies is in the form of an antibody drug conjugate, wherein the EGFR antibody pair is selected from:

992+1024, specifically including the case where a toxin is conjugated (i) only to 992, (ii) only to 1024, or (iii) to both 992 and 1024;

cetuximab and matuzumab where a toxin is conjugated (i) only to cetuximab, (ii) only to matuzumab, or (iii) to both antibodies;

cetuximab and 111 where a toxin is conjugated (i) only to cetuximab, (ii) only to 111, or (iii) to both antibodies; and

111 and 565 where a toxin is conjugated (i) only to 111, (ii) only to 565, or (iii) to both antibodies.

In other embodiments, the present pharmaceutical combination comprises 3 or more EGFR antibodies, all of which are able to bind simultaneously to EGFR. In this case, at least of the three antibodies comprises a conjugated toxin. In an alternative, at least two of the antibodies are toxin-conjugated. In another embodiment, all three of the EGFR antibodies are conjugated to a toxin. The toxin is independently selected, and can be the same or different on the various antibodies.

The literature describes one such combination of three EGFR antibodies, and the present combination can include this very combination provided at least one, at least two or all three antibodies incorporates a conjugated toxin. Thus, the combination can be (with reference to the published antibody designations:

An MM-151 mixture (see WO 2011/140254 the entire contents of which are incorporated herein by reference) where a toxin is conjugated (i) only to ca, (ii) only to cd, (iii) only to ch, (iv) to any two of ca, cd and ch including ca and cd, or cd and ch, or ca and ch, or (v) to all three of ca, cd and ch. Again, the toxin is independently selected, and can be the same or different on the various antibodies.

Therapeutic formulations of each EGFR antibody drug, including any naked EGFR antibody as well as any EGFR antibody drug conjugate, can be formulated together or separately. They can also be prepared for therapeutic use directly or for storage by mixing the conjugate having the desired degree of purity with optional pharmaceutically acceptable carriers, excipients or stabilizers (Remington's Pharmaceutical Sciences, 16^{th} edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagines, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

The active ingredients to be used for in vivo administration must be sterile. This is readily accomplished by filtration through sterile membranes.

The active ingredients can be combined into one formulation, or they can more preferably be formulated individually and provided in combination, for use as instructed to treat a given patient.

Sustained-release preparations may be prepared. Suitable examples of sustained-release include semipermeable matrices of solid hydrophobic polymers containing the conjugate, which matrices are in the form of shapes articles, e.g., films or microcapsules. Examples of sustained-release matrices include polyesters, hydrogels (for example, poly (2-hydroxyethyl-methacrylate), polylactides, copolymers of L-glutamic acid and ethyl-L-glutamate, non-degradable ethylene-vinyl acetate, degradable lactic acid-glycolic acid copolymers such as injectable microspheres composed of lactic acid-glycolic acid copolymer and leuprolide acetate, and poly-D-(−)-3-hydroxybutyric acid. While polymers such as ethylene-vinyl acetate and lactic acid-glycolic acid enable release of molecules for over 100 days, certain hydrogels release proteins for shorter time periods.

The combination of naked and/or conjugated EGFR antibody species is useful to treat EGFR+ disease cells. Such treatment results in a reduction in the number, size or distribution of such disease cells in subjects presenting with them. In embodiments, the conjugates are used to treat EGFR+ disease cells that are EGFR+ cancer cells and tumours comprising them. Such treatment results preferably in a reduction in the number, size, volume or distribution of such cancer cells and tumours comprising them, or at least in a reduction in the rate at which such disease cells increase in number, size, volume or distribution of such cells and tumours in subjects presenting with them. Thus, an effective antibody or ADC is one that will affect an EGFR+ disease cell to cause one or more of (i) reduced EGFR signalling, (ii) reduced cell viability revealed as increased killing, (iii) induced apoptosis, and (iv) inhibited proliferation. Assays are well established to measure all of these endpoints.

Subjects presenting with EGFR+ cancer cells can be identified with the aid of assays that detect the receptor, as protein or as nucleic acid precursor (DNA or RNA) in physiological samples such as biopsied tissue. A suitable test for EGFR protein is the commercially available and FDA approved Dako EGFR pharmDx® test kit.

For the treatment of subjects presenting with EGFR+ cancer cells, the appropriate dosage of the combination will depend on the type of disease to be treated, as defined above, the severity and course of the disease, whether the agent is administered for preventative or therapeutic purposes, previous therapy, the patients clinical history and response to the agent, and the discretion of the attending physician. The agent is suitably administered to the patient at one time or over a series of treatments.

The subject can be treated to introduce the antibody combination endogenously by administered each EGFR antibody drug in succession, together or separately. When the drugs are administered separately, it is desirable that they are present together within the subject so that each can exert its effect simultaneously within the patient.

For example, depending on the type and severity of the disease, about 1 μg/kg to 15 mg/kg (e.g., 0.1-20 mg/kg) of each antibody drug is a candidate dosage for administration to the patient, whether, for example, by one or more separate administrations, or by continuous infusion. A typical daily dosage might range from about 1 μg/kg to 500 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

It will thus be appreciated that an effective amount of the drug combination is an amount effective as part of a treatment regimen that retards or inhibits the rate of growth or proliferation of EGFR+ disease cells, or that otherwise alters EGFR+ disease cells beneficially.

An EGFR+ disease cell is a disease cell that presents EGFR on its surface as detectable for instance by EGFR antibody binding, or by detection of intracellular mRNA encoding her-1. Particular EGFR+ disease cells include those having on their surface an abnormally high density and/or activity of EGFR molecules, or the presence of the EGFRvIII variant of EGFR.

It may be useful to administer each of the antibody drugs by intravenous infusion first as loading dose, followed by maintenance dose, such as at an initial dose of 4 mg/kg over 90 minutes, then 2 mg/kg over 30 minutes, once weekly for as many as 52 weeks, with follow up as required. In the specific case of the panitumumab conjugate, dosing might be based on that utilized for panitumumab per se, which comprises 6 mg/kg given once every two weeks as a one hour infusion.

The antibody drug combination is useful in the treatment of a variety of cancers, to inhibit the growth or proliferation of EGFR+ cancer cells and tumours comprising them, including hematopoietic cell cancers and solid tumours. Conditions or disorders to be treated include benign or malignant tumors (e.g., renal, liver, kidney, bladder, breast, gastric, ovarian, colorectal, prostate, pancreatic, lung, vulva, and thyroid); hepatic carcinomas; sarcomas; glioblastomas; and various head and neck tumors; leukemias and lymphoid malignancies. In particular embodiments, the antibody or bivalent fragment are used in the treatment of such cancer cells that express EGFRvIII, as determined by the screening assays herein described. In particular embodiments, the cancer cells are EGFR+-presenting cancer cells that include head and neck cancers and especially squamous cell carcinoma of the head and neck, colorectal cancers, gastrointestinal cancers, brain tumours including glioblastomas, and tumours of the lung including non-small-cell lung carcinoma, and of the breast, pancreas, esophagus, kidney, ovary, cervix and prostate. In specific embodiments, the EGFR+ cancer is one for which cetuximab has received FDA marketing approval, such as squamous cell carcinoma of the head and neck and colorectal cancers.

It will be appreciated that subjects who could benefit from the present method include humans as well as other mammals such as livestock, and pets.

Still other therapeutic regimens may be combined with the administration of the antibody drug combination of the instant invention. For example, the patient to be treated may also receive radiation therapy, such as external beam radiation. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the EGFR antibody drug combination, or may be given simultaneously therewith. The conjugate may be combined with any anti-cancer toxins, or any other suitable drug particularly including irinotecan (CPT-11), cisplatin, cyclophosphamide, melphalan, dacarbazine, doxorubicin, daunorubicin, and topotecan, as well as tyrosine kinase inhibitors, including particularly EGFR kinase inhibitors such as AG1478 ((4-(3-chloroanilino-6,7-dimethoxyquinazoline), gefitinib (Iressa®), erlotinib (Tarceva®), lapatinib (Tykerb®), canertinib (PD183805, Pfizer), PKI-166 (Novartis), PD158780 and pelitinib.

It may also be desirable to administer antibodies or conjugates against other tumor associated antigens or their ligands, such as antibodies which bind to the ErbB2 (including trastuzumab marketed as Herceptin®, and pertuzumab marketed as Omnitarg®), ErbB3, ErbB4, or vascular endothelial factor (VEGF), and/or antibodies that bind to EGF or TGFα.

In another embodiment of the invention, an article of manufacture containing the EGFR antibody drug combination in an amount useful for the treatment of the disorders described herein is provided. The article of manufacture comprises one or both EGFR antibody drugs of the present antibody drug combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used in combination with another EGFR antibody drug in accordance with the present invention, thereby to achieve a synergistic effect on the EGFR+ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

An anti-cancer EGFR antibody drug according to the invention may be administered with a pharmaceutically-acceptable diluent, carrier, or excipient, in unit dosage form. Unit doses are suitably 50 mgs, 100 mgs, 150 mgs, 200 mgs, 250 mgs, 300 mgs and 400 mgs. The drug can be formulated in single use vials at a concentration such as 20 mg/mL, for instance 100 mg in 5 mL vehicle such as 0.9% saline, 200 mg in 10 mL or 400 mg in 20 mL.

Similarly, an anti-cancer naked EGFR antibody according to the invention may be administered with a pharmaceutically-acceptable diluent, carrier, or excipient, in unit dosage form. Unit doses are suitably 50 mgs, 100 mgs, 150 mgs, 200 mgs, 250 mgs, 300 mgs and 400 mgs. The drug can be formulated in single use vials at a concentration such as 20 mg/mL, for instance 100 mg in 5 mL vehicle such as 0.9% saline, 200 mg in 10 mL or 400 mg in 20 mL.

Any appropriate route of administration can be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, pulmonary, or oral administration. Different routes of administration can be used to administer the different EGFR antibody drugs.

### Example 1—Preparation of Antibodies 992 and 1024

These antibodies and their synthesis, as well as antibody 1030 and related other antibodies, are described by Symphogen et al in U.S. Pat. No. 7,887,805, the entire contents of which are incorporated herein by reference. These antibodies have a variable region that, for the 992 antibody, has SEQ ID No.24 for the heavy chain and SEQ ID No. 25 for the light chain. For the 1024 antibody, the variable region of the light chain has SEQ ID No.27, and for the heavy chain has SEQ ID No.26. These antibodies can be produced as taught by Symphogen. Similarly, other antibodies useful in the present combination can be produced in a like manner, and using for guidance the sequence information that is reproduced herein, and is otherwise available to the public.

### Example 2—Preparation of Antibody Drug Conjugates

Preparation of antibody drug conjugates is achieved using methods established in the art. In one example, cetuximab is first produced as described in AvidBiologics' WO 2012/100346, and is then conjugated to the maytansinoid DM1 using, for instance, the non-cleavable heterobifunctional cross-linking reagent SMCC.

More particularly, the antibody is buffer exchanged into 50 mM potassium phosphate, 50 mM sodium chloride, 2 mM EDTA; pH 6.5 buffer (Buffer A). All buffers in this experiment were tested to be free of endotoxin using a chromogenic Limulus amoebocyte lysate (LAL) method (Cambrex). The concentration of antibody was measured using an extinction coefficient of 1.45 mL/mg/cm at 280 nm and a molecular weight of 145,781 g.

### B. Preparation and Measurement of SMCC Stock Solution

A 20 mM solution of SMCC (6.69 mg/mL) (Concortis Biosystems Corp.) was prepared in DMSO. The solution was diluted 1/40 in Assay Buffer and the absorbance of the samples was measured at 302 nm. The concentration of the stock solution was calculated using a molar extinction coefficient of 602/M/cm.

### C. Preparation and Measurement of DM-1 Stock Solution

A 10 mM solution of DM1 (free thiol form; Concortis Biosystems Corp.) was prepared in DMA (7.37 mg/mL). The absorbance of dilutions of the stock solution in ethanol was measured at 280 nm. The concentration of stock DM1 was calculated by using a molar extinction coefficient of 5700/M/cm at 280 nm. The concentration of free —SH in the stock DM1 preparation was measured using Elman's reagent (DTNB). Dilutions of the stock solution were prepared in Assay buffer made to 3% (v/v) DMA, and then 100 mM DTNB in DMSO (1/100th volume) was added. The increase in absorbance at 412 nm was measured against a reagent blank and the concentration was calculated using an extinction coefficient of 14150/M/cm. The concentration of —SH resulting from the Elman's assay was used to represent the DM1 stock concentration in calculations for conjugation conditions.

D. Modification of Antibody with SMCC Crosslinker

Each antibody was modified using a 7.5-fold molar excess of SMCC at 20 mg/mL antibody. The reaction was carried out in Buffer A (95% v/v) with DMSO (5% v/v) for 2 hours at room temperature with stirring.

### E. G25 Chromatography to Remove Excess SMCC

The antibody-SMCC reaction mixture was gel-filtered through a 1.5×4.9 cm pre-packed column of Sephadex G25 resin equilibrated in Buffer A. The load and elution volumes were according to manufacturer's instructions (Amersham Biosciences). The concentration of the modified antibody solution was assayed spectrophotometrically using the extinction co-efficient described above.

F. Conjugation of Antibody-SMCC with DM1

The modified antibody was reacted with a 1.7-fold excess of DM1 over linker (assuming 5 linkers per antibody). The reaction was carried out at 10 mg/mL antibody concentration in Buffer A (94% v/v) with DMA (6% v/v). After addition of DM1, the reaction was incubated at room temperature for 16.5 hours with stirring.

### G. Conjugation Purification by G25 Chromatography

The conjugation reaction mixture was gel-filtered through a 1.5×4.9 cm pre-packed column of Sephadex G25 resin equilibrated in 1×phosphate buffered saline (PBS), pH 6.5 (Buffer B). The load and elution volumes were according to manufacturer's instructions (Amersham Biosciences). The number of DM1 molecules linked per mole of cetuximab was determined by measuring absorbance at both 252 nm and 280 nm of the eluted material. The DM1/antibody ratio was found to be 2 and 4. The resulting conjugate was analyzed for binding and cytotoxicity.

### Example 3—Testing of EGFR Antibody Drug Combinations

The cell lines used in these studies have the following characteristics:

A549: lung cell carcinoma cell line available at ATCC; plated at 4,000 cells/well in RPMI-10% FBS, 100 ul/well in 96 well plates.

NCI-H292: lung squamous cell carcinoma cell line; available at ATCC; plated at 4000 cells/well in RPMI-10% FBS, 100 μl/well in 96-well culture plate.

NCI-H226: lung squamous cell carcinoma cell line; available at ATCC; plated at 4000 cells/well in RPMI-10% FBS, 100 μl/well in 96-well culture plate.

### Experimental BioAssay Protocols

Each anti-EGFR antibody drug conjugate (singly or in 50:50 mixture) was added at increasing concentration to cells (as indicated) and incubated for 5 days at 37° C. Effects were then evaluated on cell growth/survival (sulforhodamine B). Results are tabulated below:

TABLE 1
Summary IC50 values for ADC and ADC mixture.

| | | | | | 992- | 992- |
|---|---|---|---|---|---|---|
| | | | | | DM1 + | DM1 + |
| | 992- | 992- | 1024- | 1024- | 1024- | 1024- |
| | DM1 | DM1 | DM1 | DM1 | DM1 | DM1 |
| | IC50 | Span | IC50 | Span | IC50 | Span |
| cell line | (nM) | (%) | (nM) | (%) | (nM) | (%) |
| A549 | 2.2 | 87 | 24.7 | 114 | 0.11 | 78 |
| NCI-H292 | 0.22 | 83 | 0.09 | 76 | 0.03 | 83.9 |
| NCI-H226 | 1.07 | 75.6 | 1.89 | 79 | 0.13 | 71 |

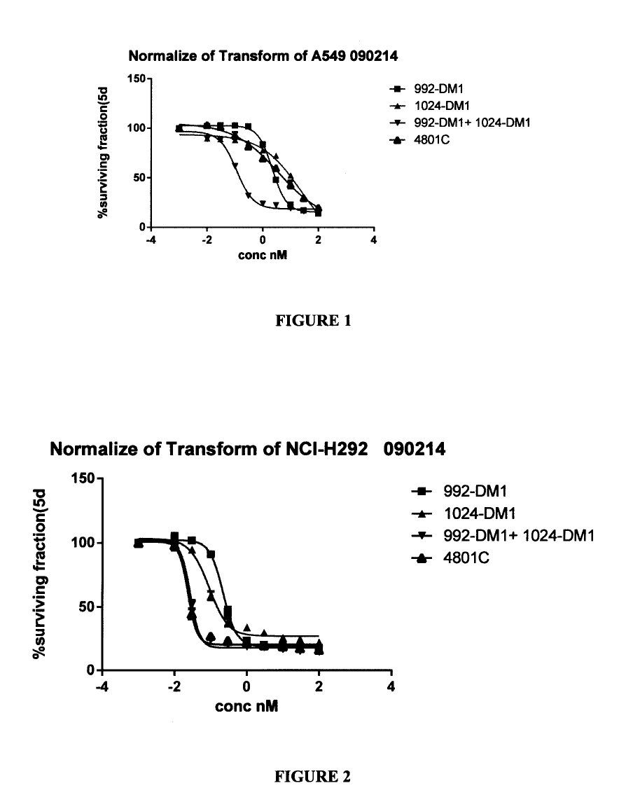

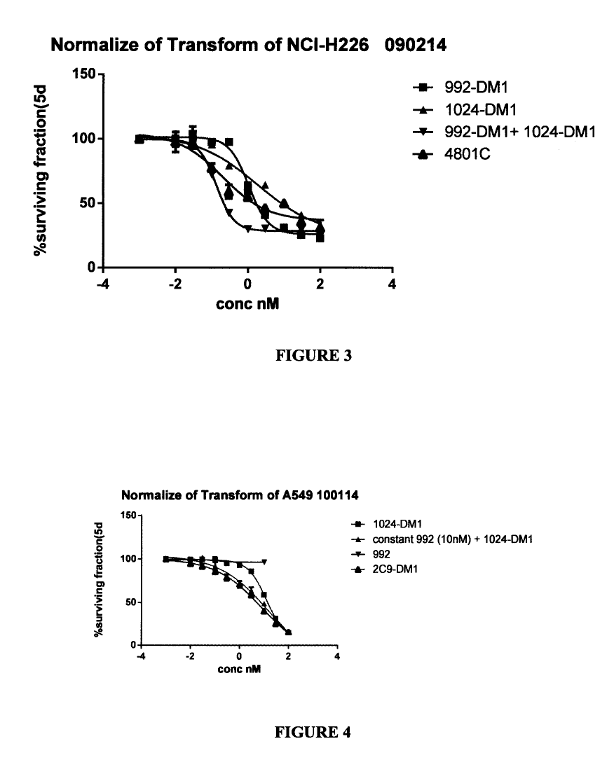

The potency and efficacy of individual ADCs and ADC mixtures in inhibiting cancer cell growth was investigated in A549, NCI-226, and NCI-H292 cancer cell lines. The results presented in FIGS. 1, 2, and 3 show that the ADC mixture is synergistic because it is several fold more potent at inhibiting the growth of these cancer cell lines compared to individual ADCs. Anti-EGFR ADC 4801C was used as a positive control. The IC₅₀ values of the individual ADCs and ADC mixture are summarized in Table 1.

It was further demonstrated that a mixture containing an ADC and naked anti-EGFR antibody to a non-overlapping EGFR epitope also acts in a synergistic manner. FIG. 4 demonstrates that while individually, 1024-DM1 ADC has some anticancer activity and unconjugated antibody 992 demonstrates no anticancer activity at the concentrations tested, combining the two agents creates a mixture that is more potent than the individual components.

Based on these findings, it is now demonstrated that combining two different species of EGFR antibody, as drug conjugates, that bind to non-overlapping EGFR epitopes results in synergistic anti-cancer activity of the two ADCs. This synergistic anti-cancer activity is superior to the activity of the corresponding naked anti-EGFR antibody mixture. Similarly, mixture of the ADCs based on anti-EGFR antibodies possess superior synergistic activity compared each individual anti-EGFR ADC in the mixture at equimolar concentration. Moreover, anti-cancer activity of the anti-EGFR ADC mixture is observed in EGFR expressing cell lines that are not very sensitive or are resistant to killing by the naked antibody mixture and any individual anti-EGFR ADCs contained in the mixture.

It is thus in accordance with the present invention that human subjects are treated with a combination of at least two different EGFR antibody species, wherein at least one and preferably two of those species are provided as ADCs, when those subjects present with EGFR+ disease cells.

All references cited herein, including all database references and the sequence information referenced therein, and are hereby incorporated herein in their entirety.

1. In combination, a first EGFR antibody species and a second EGFR antibody species, wherein both of said species can bind simultaneously to EGFR and further wherein at least one of said EGFR antibody species is conjugated to a cytotoxin and cooperates synergistically with the second EGFR antibody species.

2. The combination according to claim 1, wherein the first EGFR antibody species is a naked EGFR antibody species.

3. The combination according to claim 1, wherein the at least two EGFR antibody species are conjugated to the same or different types of cytotoxin.

4. The combination according to claim 1, comprising a first EGFR antibody species and a second EGFR antibody species and a third EGFR antibody species, wherein all of said species can bind simultaneously to EGFR and further wherein at least one of said EGFR antibody species is conjugated to a cytotoxin and cooperates synergistically with the second EGFR antibody species and the third EGFR antibody species.

5. The combination according to claim 1, wherein each EGFR antibody species is different and is selected independently from cetuximab, cetuximab variants, panitumumab, necitumumab, zalutumumab, matuzumab, nimotuzumab, ch806, species 13.1, species 13.1.2, antibodies comprised within the SYM004 mixture that includes 992 and 1024 and 1030, species 111, species 565 as well as the antibody species within the MM-151 mixture that includes antibodies designated ca, cd, ch, and EGFR antibody species J2898A.

6. The combination according to claim 5, comprising antibody species 992 and 1024, wherein at least one of said species comprises a conjugated cytotoxin.

7. The combination according to claim 5, comprising antibody species 992 and 1024, wherein 1024 comprises a conjugated cytotoxin and 992 is a naked antibody species.

8. The combination according to claim 5, comprising antibody species 992 and 1024, wherein 1024 is a naked antibody species and 992 comprises a conjugated cytotoxin.

9. The combination according to claim 5, comprising antibody species 992 and 1024, wherein both 1024 and 992 comprise a conjugated cytotoxin.

10. The combination according to claim 5, comprising antibody species cetuximab.

11. The combination according to claim 10, comprising cetuximab conjugated with a cytotoxin.

12. The combination according to claim 5, comprising antibody species panitumumab.

13. The combination according to claim 12, comprising panitumumab conjugated with a cytotoxin.

14. The combination according to claim 1, wherein the cytotoxin can be the same or different and is an anti-microtubule toxin.

15. The combination according to claim 14, wherein the anti-microtubule toxin is selected from a maytansinoid, a dolostatin, an auristatin, a tubulysin, and a cryptophycin.

16. The combination according to claim 15, wherein the cytotoxin is DM-1.

17. The combination according to claim 15, wherein the cytotoxin is an auristatin.

18. The combination according to claim 10, comprising cetuximab linked to DM-1 by an SMCC linker.

19. The combination according to claim 6, comprising antibodies species 992 linked to DM-1 by an SMCC linker.

20. (canceled)

21. The combination according to claim 6, comprising antibody species 1024 linked to DM-1 by an SMCC linker.

22. In a method for treating a subject presenting with EGFR+ disease cells using a pharmaceutical combination of two or more naked EGFR antibody species, the improvement wherein the pharmaceutical combination comprises at least one EGFR antibody species that has been conjugated to a cytotoxin thereby to produce an antibody drug conjugate that cooperates synergistically with the other EGFR antibody species to inhibit growth of said disease cells.

23. A method for controlling the growth of EGFR+ disease cells, comprising administering to a subject in need thereof, a pharmaceutical combination comprising at least two EGFR antibodies that bind EGFR simultaneously, wherein at least one of said EGFR antibodies is provided as an antibody-drug conjugate, and further wherein the effect of the drug combination is synergistic with respect to the killing of said disease cells.

24. The method according to claim 22, wherein the pharmaceutical composition comprises a first EGFR antibody species and a second EGFR antibody species, wherein both of said species can bind simultaneously to EGFR and further wherein at least one of said EGFR antibody species is conjugated to a cytotoxin and cooperates synergistically with the second EGFR antibody species.

25. (canceled)

26. The method according to claim 23, wherein the pharmaceutical composition comprises a first EGFR antibody species and a second EGFR antibody species, wherein both of said species can bind simultaneously to EGFR and further wherein at least one of said EGFR antibody species is conjugated to a cytotoxin and cooperates synergistically with the second EGFR antibody species.

27. The method according to claim 26, wherein the EGFR+ disease cells are cancer cells.
